(19) United States
(12) Patent Application Publication
(10) Pub. No.: US 2023/0293417 A1
(43) Pub. Date: Sep. 21, 2023

(54) ADENOSINE-CONTAINING COMPOSITION AND METHOD FOR SUPPRESSING PRECIPITATION OF ADENOSINE

(71) Applicant: Shiseido Company, Ltd., Tokyo (JP)

(72) Inventor: Keiko NAKAMURA, Tokyo (JP)

(73) Assignee: Shiseido Company, Ltd., Tokyo, (JP)

(21) Appl. No.: 18/020,227

(22) PCT Filed: Aug. 16, 2021
(86) PCT No.: PCT/JP2021/029928

§ 371 Date: Feb. 7, 2023

(30) Foreign Application Priority Data
Sep. 1, 2020 (JP) 2020-146939
Jan. 12, 2021 (JP) 2021-002813
Jul. 26, 2021 (JP) 2021-121685

(51) Int. Cl. A61K 8/60; A61K 8/73; A61K 8/34

(52) U.S. Cl. CPC A61K 8/606; A61K 8/34; A61K 8/73

(57) ABSTRACT

An adenosine-containing composition contains (A) adenosine, (B) at least one selected from the group consisting of salicylic acid and salts thereof, glycyrrhizic acid and salts thereof, and plant extracts, and (C) water. The amount of ethanol is less than 20% by mass relative to the mass of the adenosine-containing composition.

### CROSS REFERENCE TO RELATED APPLICATIONS

The present application is based upon and claims the benefit of priority from Japanese Patent Application No. 2020-146939 filed on Sep. 1, 2020, Japanese Patent Application No. 2021-2813 filed on Jan. 12, 2021, and Japanese Patent Application No. 2021-121685 filed on Jul. 26, 2021, the entire contents of which are incorporated herein by reference.

### TECHNICAL FIELD

The present disclosure relates to a composition containing adenosine. The present disclosure relates to a method for suppressing the precipitation of adenosine in an aqueous solvent.

### BACKGROUND ART

Adenosine is known to have a blood circulation-promoting effect when applied to the skin. Adenosine is also known to have an effect of producing a hair growth factor in dermal papilla cells. Hence, adenosine is used in external-use skin preparations such as hair restorers (see, for example, Patent Literature 1).

The external-use composition disclosed in Patent Literature 1 contains: 1.0-2.0 mass% of adenosine to the entire composition; 10.0-20.0 mass% of a moisturizer to the entire composition, wherein, in the moisturizer, the content of a sugar-based moisturizer is 20.0-100 mass% to the entire moisturizer; and 35.0-65.0 mass% of a lower alcohol to the entire composition.

### CITATION LIST

### Patent Literature

Patent Literature 1: Japanese Unexamined Patent Publication No. 2008-247752

### SUMMARY OF INVENTION

### Technical Problem

The following analysis can be made from the perspective of the present disclosure.

To make an adenosine-containing composition, as disclosed in Patent Literature 1, effectively act on the skin, adenosine needs to be dissolved in a solvent. Adenosine, however, has low solubility to water. Therefore, in the composition of Patent Literature 1, at least 30% by mass of ethanol (typically referred to as “alcohol”) is used with respect to the mass of the composition to dissolve the adenosine in an aqueous solvent. Unfortunately, there are people who, when ethanol is applied to the skin, get skin disorders or have an allergic reaction to ethanol. Hence, there is a demand for adenosine-containing products with low ethanol content for such people who cannot apply ethanol to the skin. Also, in recent years, there has been an increase in demand for alcohol-free external-use skin preparations.

There are cases where adenosine can be dissolved in an aqueous solvent, even when the ethanol concentration in the aqueous solvent is low. However, when the composition is temporarily placed in a low-temperature environment (e.g., 0° C.), it is impossible to maintain the dissolved state of adenosine simply by lowering the ethanol concentration. For example, if the ethanol concentration is lowered in the composition disclosed in Patent Literature 1, adenosine can be dissolved provisionally at a room-temperature environment (e.g., 25° C. or higher), but the adenosine cannot be kept in a dissolved state and will precipitate in cases where the adenosine solution is temporarily placed in a low-temperature environment (e.g., 0° C.). Precipitation of adenosine will cause no problem if the adenosine can be re-dissolved when the solution is returned to a room-temperature environment (e.g., 25° C. or higher). Unfortunately, in cases where the ethanol concentration in a composition is simply lowered, the precipitated adenosine cannot be re-dissolved even when the composition is returned to a room-temperature environment. Hence, it is desired that, even in cases where the ethanol concentration in a solvent is low, adenosine does not precipitate out even when the adenosine solution is temporarily in a low-temperature state, or the precipitated adenosine can be re-dissolved spontaneously when returned to an environment at 25° C. or higher.

As described above, there is a demand for an adenosine-containing composition in which precipitation of adenosine can be suppressed at low temperatures even when the ethanol content is low, or in which the adenosine’s dissolved state can be ensured at room temperature. There is also a demand for a method for suppressing precipitation of dissolved adenosine even in an aqueous solvent having a low ethanol content, and/or a method for facilitating re-dissolution of precipitated adenosine.

### Solution to Problem

According to a first aspect of the present disclosure, an adenosine-containing composition is provided, the composition comprising: (A) adenosine; (B) at least one selected from the group consisting of salicylic acid and salts thereof, glycyrrhizic acid and salts thereof, and plant extracts; and (C) water. A content of ethanol is less than 20% by mass relative to the mass of the adenosine-containing composition.

According to a second aspect of the present disclosure, a method for suppressing precipitation of adenosine is provided, the method comprising: dissolving adenosine and a first component to an aqueous solvent containing less than 20% by mass of ethanol. The first component is at least one selected from the group consisting of salicylic acid and salts thereof, glycyrrhizic acid and salts thereof, and plant extracts.

### Advantageous Effects of Invention

In the adenosine-containing composition of the present disclosure, adenosine is dissolved in an aqueous solvent in an environment at 25° C. or higher, even though the content by percentage of ethanol is low. Further, in the adenosine-containing composition of the present disclosure, adenosine is less likely to precipitate even when the ambient environment is at low temperatures, and also, even if adenosine precipitates at low temperatures, the adenosine can dissolve spontaneously by raising the ambient environment temperature.

According to a method for suppressing precipitation of adenosine of the present disclosure, adenosine can be dissolved in an aqueous solvent having a low content by percentage of ethanol, and also, precipitation of the dissolved adenosine can be suppressed. Further, even if adenosine precipitates due to a drop in ambient environment temperature, the adenosine can dissolve spontaneously by raising the ambient environment temperature.

### DESCRIPTION OF EMBODIMENTS

Preferred modes of the aforementioned aspects will be described below.

According to a prefered mode of the aforementioned first aspect, the plant extract is at least one selected from the group consisting of Angelica keiskei leaf/stem extract, Prunus armeniaca (apricot) juice, Ginkgo biloba leaf extract, Citrus unshiu peel extract, Scutellaria baicalensis root extract, Panax ginseng root extract, Rosa canina fruit extract, Ononis spinosa extract, Glycyrrhiza glabra (licorice) root extract, Phellodendron amurense bark extract, Sophora angustifolia root extract, Prunus speciosa leaf extract, Hypericum perforatum flower/leaf/stem extract, Crataegus monogyna flower extract, Aesculus hippocastanum (horse chestnut) seed extract, Achillea millefolium extract, Rosa centifolia flower extract, Prunus yedoensis leaf extract, Camellia japonica seed extract, Camellia japonica flower extract, Rubus suavissimus (raspberry) extract, Nelumbo nucifera germ extract, Syzygium jambos leaf extract, Bupleurum falcatum root extract, Melilotus officinalis extract, Centaurea cyanus flower extract, Citrus limon (lemon) extract, Rosmarinus officinalis (rosemary) extract, Rosmarinus officinalis (rosemary) leaf extract, Thymus serpyllum extract, and Sanguisorba officinalis extract.

According to a prefered mode of the aforementioned first aspect, a content by percentage of the component (A) relative to the mass of the adenosine-containing composition is from 5×10⁻⁴% by mass to 8% by mass.

According to a prefered mode of the aforementioned first aspect, the component (B) is a component which suppresses precipitation of the dissolved component (A).

According to a prefered mode of the aforementioned first aspect, the component (B) further contains caffeine.

According to a prefered mode of the aforementioned first aspect, a content by percentage of the component (B) relative to the mass of the adenosine-containing composition is 1×10⁻⁴% by mass or greater.

According to a prefered mode of the aforementioned first aspect, a content by percentage of the component (C) relative to the mass of the adenosine-containing composition is 60% by mass or greater.

According to a prefered mode of the aforementioned first aspect, the composition further comprises from 5% by mass to 40% by mass of a polyol relative to the mass of the adenosine-containing composition.

According to a prefered mode of the aforementioned first aspect, the composition is a single-phase liquid composition. In atmospheric pressure at 25° C., the component (A) and the component (B) are dissolved in the adenosine-containing composition.

According to a prefered mode of the aforementioned first aspect, the composition comprises substantially no ethanol.

According to a prefered mode of the aforementioned first aspect, the composition is applicable in an external-use skin preparation applicable to the skin.

According to a prefered mode of the aforementioned first aspect, the composition is an external-use skin preparation for application to the scalp.

According to a prefered mode of the aforementioned second aspect, in the aqueous solvent, a content of water relative to the mass of the aqueous solvent is 60% by mass or greater.

According to a prefered mode of the aforementioned second aspect, the plant extract is at least one selected from the group consisting of Angelica keiskei leaf/stem extract, Prunus armeniaca (apricot) juice, Ginkgo biloba leaf extract, Citrus unshiu peel extract, Scutellaria baicalensis root extract, Panax ginseng root extract, Rosa canina fruit extract, Ononis spinosa extract, Glycyrrhiza glabra (licorice) root extract, Phellodendron amurense bark extract, Sophora angustifolia root extract, Prunus speciosa leaf extract, Hypericum perforatum flower/leaf/stem extract, Crataegus monogyna flower extract, Aesculus hippocastanum (horse chestnut) seed extract, Achillea millefolium extract, Rosa centifolia flower extract, Prunus yedoensis leaf extract, Camellia japonica seed extract, Camellia japonica flower extract, Rubus suavissimus (raspberry) extract, Nelumbo nucifera germ extract, Syzygium jambos leaf extract, Bupleurum falcatum root extract, Melilotus officinalis extract, Centaurea cyanus flower extract, Citrus limon (lemon) extract, Rosmarinus officinalis (rosemary) extract, Rosmarinus officinalis (rosemary) leaf extract, Thymus serpyllum extract, and Sanguisorba officinalis extract.

According to a prefered mode of the aforementioned second aspect, from 5×10⁻⁴% by mass to 8% by mass of adenosine is added to a total mass of composition.

According to a prefered mode of the aforementioned second aspect, the first component is a component which suppresses precipitation of the dissolved adenosine.

According to a prefered mode of the aforementioned second aspect, the first component further contains caffeine.

According to a prefered mode of the aforementioned second aspect, 1×10⁻⁴% by mass or greater of the first component is added to the total mass of the composition.

According to a prefered mode of the aforementioned second aspect, the method further comprises adding, to the aqueous solvent, from 5% by mass to 40% by mass of a polyol relative to the total mass of the composition.

According to a prefered mode of the aforementioned second aspect, a single-phase liquid composition in which component (A) and component (B) have been dissolved is prepared.

According to a prefered mode of the aforementioned second aspect, the method does not comprise adding ethanol.

In the present disclosure, “substantial amount” refers to an amount capable of bringing about the functions/effects achieved by the addition of the compound in question.

In the following description, POE is an acronym of polyoxyethylene, POP is an acronym of polyoxypropylene, and the number in parentheses after POE or POP indicates the average number of moles of POE groups or POP groups added in the compound in question.

An adenosine-containing composition according to a first embodiment of the present disclosure will be described. An adenosine-containing composition of the present disclosure contains (A) adenosine, (B) at least one selected from the group consisting of caffeine, glycyrrhizic acid and salts thereof, salicylic acid and salts thereof, and plant extracts, and (C) water. The adenosine-containing composition is a liquid composition. It is preferred that the adenosine-containing composition is a single-phase aqueous composition. The adenosine-containing composition may be an emulsion containing an oily component.

### (A) Adenosine

It is preferred that adenosine is dissolved in the adenosine-containing composition at atmospheric pressure at 25° C. (or in a state where the composition is left to stand preferably at 25° C. for 4 or more weeks after the adenosine has precipitated in a low-temperature environment). Whether or not the adenosine is dissolved can be determined by visually observing the appearance.

The content by percentage of adenosine relative to the mass of the adenosine-containing composition is preferably 5×10⁻⁴% by mass or greater. The content by percentage of adenosine relative to the mass of the adenosine-containing composition may be 8×10⁻⁴% by mass or greater, 1×10⁻³% by mass or greater, 5×10⁻³% by mass or greater, 1×10⁻²% by mass or greater, 5×10⁻²% by mass or greater, 0.1% by mass or greater, 0.2% by mass or greater, 0.3% by mass or greater, 0.5% by mass or greater, 0.8% by mass or greater, 1% by mass or greater, or 1.5% by mass or greater. If the content by percentage of adenosine is less than 5×10⁻⁴% by mass, the effective actions of adenosine cannot be obtained. The content by percentage of adenosine relative to the mass of the adenosine-containing composition is preferably 8% by mass or less. The content by percentage of adenosine relative to the mass of the adenosine-containing composition may be 5% by mass or less, 4% by mass or less, 3.5% by mass or less, 3% by mass or less, 2.5% by mass or less, 2% by mass or less, 1.5% by mass or less, or 1% by mass or less. If the content by percentage of adenosine exceeds 8% by mass, adenosine is likely to precipitate.

### (B) Precipitation-Suppressing Component

The adenosine-containing composition of the present disclosure contains at least one component selected from the group consisting of caffeine, glycyrrhizic acid and salts thereof, salicylic acid and salts thereof, and plant extracts. In the present disclosure, for the sake of convenience, these components are referred to as precipitation-suppressing components. A precipitation-suppressing component is a component capable of suppressing precipitation of adenosine dissolved in a solvent, or capable of helping (promoting) re-dissolution of adenosine in cases where the adenosine temporarily precipitates in the composition in a low-temperature environment (e.g., 0° C.) and the composition is then returned to 25° C. or higher. In the adenosine-containing composition of the present disclosure, the precipitation-suppressing component is dissolved in the adenosine-containing composition at atmospheric pressure at 25° C. In cases where the precipitation-suppressing component does not dissolve in the solvent, a component capable of making the precipitation-suppressing component soluble, such as a surfactant, may be added.

Examples of the plant extracts include at least one selected from the group consisting of Angelica keiskei leaf/stem extract, Prunus armeniaca (apricot) juice, Ginkgo biloba leaf extract, Citrus unshiu peel extract, Scutellaria baicalensis root extract, Panax ginseng root extract, Rosa canina fruit extract, Ononis spinosa extract, Glycyrrhiza glabra (licorice) root extract, Phellodendron amurense bark extract, Sophora angustifolia root extract, Prunus speciosa leaf extract, Hypericum perforatum flower/leaf/stem extract, Crataegus monogyna flower extract, Aesculus hippocastanum (horse chestnut) seed extract, Achillea millefolium extract, Rosa centifolia flower extract, Prunus yedoensis leaf extract, Camellia japonica seed extract, Camellia japonica flower extract, Rubus suavissimus (raspberry) extract, Nelumbo nucifera germ extract, Syzygium jambos leaf extract, Bupleurum falcatum root extract, Melilotus officinalis extract, Centaurea cyanus flower extract, Citrus limon (lemon) extract, Rosmarinus officinalis (rosemary) extract, Rosmarinus officinalis (rosemary) leaf extract, Thymus serpyllum extract, and Sanguisorba officinalis extract. The above plant extracts are described according to their cosmetic ingredient names. Plant extracts having quasi-pharmaceutical ingredient names different from the aforementioned cosmetic ingredient names may be encompassed as the aforementioned plant extracts, so long as their INCI names are the same.

The adenosine-containing composition may contain a plurality of precipitation-suppressing components according to an arbitrary combination.

It is more preferred that the precipitation-suppressing component contains at least one component selected from the group consisting of caffeine, glycyrrhizic acid and salts thereof, salicylic acid and salts thereof, Prunus armeniaca (apricot) juice, Ginkgo biloba leaf extract, Glycyrrhiza glabra (licorice) root extract, Sophora angustifolia root extract, Camellia japonica flower extract, Citrus limon (lemon) extract, Thymus serpyllum extract, and Sanguisorba officinalis extract.

Commercially available products can be used for the aforementioned plant extracts. A plant extract can be obtained by immersing the whole herb of the plant, such as the plant’s leaf, stem (including underground stem), fruit, root, etc., in an extraction solvent, and heating the same under reflux as the case may be, followed by filtration and concentration. Any solvent ordinarily used for extraction may be used for the extraction solvent; for example, an organic solvent selected from, e.g., alcohols, such as methanol, ethanol, etc., hydrous alcohols, acetone, ethyl acetate, etc., may be used singly, or a plurality of types may be used in combination, or water may be used for extraction.

The content by percentage of the precipitation-suppressing component relative to the mass of the adenosine-containing composition is preferably 1×10⁻⁴% by mass or greater. The content by percentage of the precipitation-suppressing component relative to the mass of the adenosine-containing composition may be 2×10⁻⁴% by mass or greater, 5×10⁻⁴% by mass or greater, 8×10⁻⁴% by mass or greater, 1×10⁻³% by mass or greater, 2×10⁻³% by mass or greater, 3×10⁻³% by mass or greater, 5×10⁻³% by mass or greater, 8×10⁻³% by mass or greater, 0.01% by mass or greater, 0.02% by mass or greater, 0.05% by mass or greater, or 0.07% by mass or greater. If the content by percentage of the precipitation-suppressing component is less than 1×10⁻⁴% by mass, it is not possible to obtain the effect of suppressing precipitation of adenosine. The precipitation-suppressing component can be added up to solubility. The content by percentage of the precipitation-suppressing component relative to the mass of the adenosine-containing composition may be, for example, 1.5% by mass or less, 1% by mass or less, 0.8% by mass or less, 0.5% by mass or less, 0.2% by mass or less, 0.1% by mass or less, 0.05% by mass or less, 0.02% by mass or less, 0.01% by mass or less, or 5×10⁻³% by mass or less.

For example, the content of each of caffeine, glycyrrhizic acid and salts thereof and salicylic acid and salts thereof relative to the mass of the adenosine-containing composition may be 0.01% by mass or greater, preferably 0.05% by mass or greater. The content of each of the plant extracts relative to the mass of the adenosine-containing composition may be 5×10⁻⁴% by mass or greater, preferably 1×10⁻³% by mass or greater, more preferably 2×10⁻³% by mass or greater. The content by percentage of the plant extract is a value in terms of pure content.

The content of the precipitation-suppressing component to 1 part by mass of adenosine is preferably 0.001 parts by mass or greater. The content of the precipitation-suppressing component to 1 part by mass of adenosine may be 0.002 parts by mass or greater, 0.005 parts by mass or greater, 0.008 parts by mass or greater, 0.01 parts by mass or greater, 0.05 parts by mass or greater, or 0.1 parts by mass or greater. If the precipitation-suppressing component is less than 0.001 parts by mass, the precipitation-suppressing effect will deteriorate. The content of the precipitation-suppressing component to 1 part by mass of adenosine may be 2000 parts by mass or less, 1000 parts by mass or less, 500 parts by mass or less, 200 parts by mass or less, 100 parts by mass or less, 50 parts by mass or less, 20 parts by mass or less, 10 parts by mass or less, 5 parts by mass or less, or 1 part by mass or less.

### (C) Water

Any type of water used for cosmetics, quasi-pharmaceutical products, etc., may be used for the water. It is possible to use, for example, purified water, ion-exchanged water, tap water, etc.

The content by percentage of water relative to the mass of the adenosine-containing composition may be, for example, 30% by mass or greater, 40% by mass or greater, 50% by mass or greater, 60% by mass or greater, 65% by mass or greater, 70% by mass or greater, 75% by mass or greater, or 80% by mass or greater. The content by percentage of water relative to the mass of the adenosine-containing composition may be, for example, 95% by mass or less, 90% by mass or less, 85% by mass or less, or 80% by mass or less.

### (D) Polyol

The adenosine-containing composition of the present disclosure may further contain a polyol. For polyols, it is possible to use, for example, the compounds listed below. Among such polyols, in the adenosine-containing composition of the present disclosure, it is preferred to use, for example, propylene glycol, dipropylene glycol, 1,3-butylene glycol, etc. Adding such polyol(s) can improve adenosine solubility and precipitation-suppressing effects.

The content by percentage of polyol relative to the mass of the adenosine-containing composition may be, for example, 5% by mass or greater, 10% by mass or greater, 15% by mass or greater, 20% by mass or greater, 25% by mass or greater, 30% by mass or greater, or 40% by mass or greater. The content by percentage of polyol relative to the mass of the adenosine-containing composition may be, for example, 50% by mass or less, 40% by mass or less, 35% by mass or less, 30% by mass or less, 25% by mass or less, or 20% by mass or less.

Examples of the polyol may include dihydric alcohol (such as ethylene glycol, propylen glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, octylene glycol, etc); trihydric alcohol (such as glycerin, trimethylolpropane, etc); tetrahydric alcohol (such as such as pentaerythritol such as 1,2,6-hexanetriol, etc); pentahydric alcohol (such as xylitol, etc); hexahydric alcohol (such as sorbitol, mannitol, etc); polyhydric alcohol polymer (such as diethylene glycol, dipropylene glycol, triethylene glycol, polypropylene glycol, tetraethylene glycol, diglycerin, polyethylene glycol, triglycerin, tetraglycerin, polyglycerin, etc); dihydric alcohol alkyl ethers (such as ethylene glycol monomethyl ether, ethylene glycol monoethyl ether, ethylene glycol monobutyl ether, ethylene glycol monomphenyl ether, ethylene glycol monohexyl ether, ethylene glycol mono2-methylhexyl ether, ethylene glycol isoamyl ether, ethylene glycol benzil ether, ethylene glycol isopropyl ether, ethylene glycol dimethyl ether, ethylene glycol diethyl ether, ethylene glycol dibutyl ether, etc); dihydric alcohol alkyl ethers (such as diethylene glycol monomethyl ether, diethylene glycol monoethyl ether, diethylene glycol monombutyl ether, diethylene glycol dimethyl ether, diethylene glycol diethyl ether, diethylene glycol butyl ether, diethylene glycol methylethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, etc); dihydric alcohol ether ethers (such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disaccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, etc); glycerin monoalkyl ether (such as chimyl alcohol, selachyl alcohol, batyl alcohol, etc); sugar alcohol (such as sorbitol, maltitol, maltotriose, mannitol, sucrose, erythritol, glucose, fructose, starch sugar, maltose, xylitol, starch sugar hydrogenated alcohol, etc); glycolide, tetrahydrofurfuryl alcohol; POE-tetrahydrofurfuryl alcohol; POP/POE-butyl ether; tripolyoxypropylene glycerin ether; POP-glycerin ether; POP-glycerin ether phosphoric acid; POP/POE-pentaerythritol ether; polyglycerin, and the like.

### Ethanol

In the adenosine-containing composition of the present disclosure, the content of ethanol relative to the mass of the adenosine-containing composition is less than 20% by mass, preferably 15% by mass or less, more preferably 10% by mass or less, more preferably 5% by mass or less, more preferably 3% by mass or less, more preferably 2% by mass or less, more preferably 1% by mass or less, and it is even more preferable that substantially no ethanol (0% by mass) is included in the adenosine-containing composition. By lowering the content by percentage of ethanol, it is possible to reduce stimulation to the skin. Further, the adenosine-containing composition can be made applicable to people hypersensitive to ethanol and/or people having an allergic reaction to ethanol.

If necessary, the adenosine-containing composition of the present disclosure may contain other components as appropriate, such as water-soluble alcohols, oily components, powders, anionic surfactants, cationic surfactants, amphoteric surfactants, hydrophilic nonionic surfactants, lipophilic nonionic surfactants, thickeners, moisturizers, film-forming agents, oil-soluble UV absorbers, water-soluble UV absorbers, metal ion sequestering agents, amino acids, organic amines, polymer emulsions, pH adjusters, skin nutrients, vitamins, antioxidants, antioxidant aids, perfumes, etc., in amounts that do not inhibit the effects of the present disclosure.

Examples of water-soluble alcohols may include at least one type selected from lower alcohols, polyhydric alcohols, polyhydric alcohol polymers, dihydric alcohol alkyl ethers, dihydric alcohol alkyl ethers, dihydric alcohol ether esters, glycerin monoalkyl ethers, sugar alcohols, monosaccharides, oligosaccharides, polysaccharides, and derivatives of the above.

Examples of the lower alcohol may include ethanol, propanol, isopropanol, isobutyl alcohol, t-butyl alcohol, and the like.

Examples of the monosaccharides may include at least one selected from triose (such as D-glyceryl aldehyde, dihydroxyacetone, etc); tetrose (such as D-erythrose, D-erythrulose, D-threose, erythritol, etc); pentaose (such as L-arabinose, D-xylose, L-lyxose, D-arabinose, D-ribose, D-ribulose, D-xylulose, L-xylulose, etc); hexalose (such as D-glucose, D-talose, D-psicose, D-galactose, D-fructose, L-galactose, L-mannose, D-tagatose, etc); heptose (such as aldoheptose, heptulose, etc); octose (such as octulose, etc); deoxy sugar (such as 2-deoxy-D-ribose, 6-deoxy-L-galactose, 6-deoxy-L-mannose, etc); amino sugar (such as D-glucosamine, D-galactosamine, sialic acid, amino uronic acid, muramic acid, etc); uronic acid (such as D-grucuronic acid, D-mannuronic acid, L-guluronic acid, D-garacturonic acid, L-iduronic acid, etc) and the like.

Examples of the oligosaccharide may include at least one selected from sucrose, guntianose, umbelliferose, lactose, planteose, isolignoses, α,α-trehalose, raffinose, lignoses, umbilicin, stachyose, verbascoses, and the like.

Examples of the polysaccharide may include at least one selected from cellulose, quince seed, chondroitinsulfate, starch, galactan, dermatan sulfate, glycogen, acasia gum, heparansulfate, hyaluronan, gum tragacanth, keratan sulfate, chondoroitin, xanthan gum, mucoitin sulfate, guar gum, dextran, keratosulfate, locust bean gum, succinoglycan, caronic acid, and the like.

Examples of other polyols may include at least one polyol selected from polyoxyethylene methyl glucoside (Glucam E-10) and polyoxypropylene methyl glucoside (Glucam P-10).

Examples of oily components may include liquid oils, solid fats, waxes, hydrocarbons, higher fatty acids, higher alcohol, synthetic ester oils, and silicone oils.

Examples of the liquid oil that may be used may include avocado oil, camellia oil, turtle oil, macadamia nut oil, corn oil, mink oil, olive oil, rapeseed oil, egg yolk oil, sesame oil, par chic oil, wheat germ oil, southern piece oil, castor oil, linseed oil, safflower oil, cotton seed oil, perilla oil, soybean oil, groundnut oil, brown real oil, torreya oil, rice bran oil, Chinese tung oil, Japanese tung oil, jojoba oil, germ oil, triglycerol, and the like.

Examples of the solid fat that may be used may include cacao butter, coconut oil, horse fat, hydrogenated coconut oil, palm oil, beef tallow, sheep tallow, hydrogenated beef tallow, palm kernel oil, lard, beef bones fat, Japan wax kernel oil, hardened oil, hoof oil, Japan wax, hydrogenated caster oil, and the like.

Examples of the waxes that may be used may include beeswax, candelilla wax, cotton wax, carnauba wax, bayberry wax, insect wax, spermaceti, montan wax, bran wax, lanolin, kapok wax, lanolin acetate, liquid lanolin, sugarcane wax, lanolin fatty acid isopropyl ester, hexyl laurate, reduced lanolin, jojoba wax, hardened lanolin, shellac wax, POE lanolin alcohol ether, POE lanolin alcohol acetate, POE cholesterol ether, lanolin fatty acid polyethylene glycol, POE hydrogenated lanolin alcohol ether, and the like.

Examples of the hydrocarbon oils that may be used may include liquid paraffin, ozocerite, squalane, pristane, paraffin, ceresin. squalene, vaseline, microcrystalline wax, and the like.

Examples of the higher alcohol that may be used may include linear alcohol (such as lauryl alcohol, cetyl alcohol, stearyl alcohol, behenyl alcohol, myristyl alcohol, oleyl alcohol, and cetostearyl alcohol); branched-chain alcohol (such as monostearylglycerin ether (batyl alcohol), 2-decyltetradecinol, lanolin alcohol, cholesterol, phytosterol, hexyldodecanol, isostearyl alcohol, and octyldodecanol) and the like.

Examples of the synthesis ester oils that may be used may include isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxy stearate, ethylene glycol di-2-ethyl hexanoate, di-penta erythritol fatty acid ester, N-alkyl glycol monoisostearate, neopentyl glycol dicaprate, diisostearyl malate, glyceryl di-2-heptyl undecanoate, trimethyrol propane tri-2-ethyl hexanoate, trimethyrol propane triisostearate, pentaerythritol tetra-2-ethyl hexanoate, glyceryl tri-2-ethyl hexanoate, glyceryl trioctanoate, glyceryl triisopalmitate, trimethyrol propane triisostearate, cetyl 2-ethylhexanoate, 2-ethylhexyl palmitate, glyceryl trimyristate, glyceride tri-2-heptyl undecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, and the like.

Examples of the silicone oil may include silicone compounds such as dimethylpolysiloxane, methylhydrogenpolysiloxane, methylphenylpolysiloxane, stearoxymethylpolysiloxane, polyether-modified organopolysiloxane, fluoroalkyl/polyoxyalkylene co-modified organopolysiloxane, alkyl-modified organopolysiloxane, terminal-modified organopolysiloxane, fluorine-modified organopolysiloxane, amino-modified organopolysiloxane, silicone gel, acrylic silicone, trimethylsiloxysilicic acid, silicone RTV rubber, cyclopentasiloxane and the like.

Examples of the powder may include inorganic powder (such as talc, kaolin, mica, sericite, muscovite, phlogopite, synthetic mica, lepidolite, biotite, vermiculite, magnesium carbonate, calcium carbonate, aluminum silicate, barium silicate, calcium silicate, magnesium silicate, strontium silicate, tungstate, magnesium, silica, zeolite, glass, barium sulfate, calcined calcium sulfate (calcined gypsum), calcium phosphate, fluorine apatite, hydroxyapatite, ceramic powder, metallic soap (such as zinc myristate, calcium palimitate, and aluminum stearate), and boron nitride, etc); organic powder (such as polyamide resin powder (nylon powder), polyethylene powder, polymethylmethacrylate powder, polystyrene powder, styrene-acrylic acid copolymer powder, benzoguanamine resin powder, poly(tetrafluroethylene) powder, and cellulose powder, silicone resin powder, silk powder, wool powder, urethane powder, etc); inorganic white family pigment (such as titanium dioxide, zinc oxide, etc); inorganic red family pigment (such as iron oxide (colcothar), iron titanate, etc); inorganic brown family pigment (such as γ-iron oxide, etc); inorganic yellow family pigment (such as yellow iron oxide, loess, etc); inorganic black family pigment (such as black iron oxide, carbon black, lower titanium oxide, etc); inorganic purple family pigment (such as manganese violet, cobalt violet, etc); inorganic green family pigment (such as chrome oxide, chrome hydroxide, cobalt titanate, etc); inorganic blue family pigment (such as ultramarine, iron blue, etc); pearl pigment (such as titanium oxide coated mica, titanium oxide coated bismuth oxychloride, titanium oxide coated talc, colored titanium oxide coated mica, bismuth oxychloride, argentine, etc); metal powder pigment (such as aluminum powder, copper powder, etc); organic pigment such as zirconium, barium, or aluminum lake (such as organic pigment such as Red No.201, Red No.202, Red No.204, Red No.205, Red No.220, Red No.226, Red No.228, Red No.405, Red No.201, Orange No.203, Orange No.204, Yellow No.205, Yellow No.401, Blue No.401, Red No.3, Red No.104, Red No.106, Red No.227, Red No.230, Red No.401, Red No.505, Orange No.205, Yellow No.4, Yellow No.5, Yellow No.202, Yellow No.203, Green No.3, and Blue No.1, etc); natural pigment (such as chlorophyll, β-carotene, etc); and the like.

In cases where there are components (except for adenosine), such as precipitation-suppressing components, perfumes, etc., that do not dissolve in an aqueous solvent, a surfactant may be added to dissolve these components. The surfactant may be added in an amount necessary for such components insoluble to an aqueous solvent to be dissolved into the solvent. From the viewpoint of reducing stimulation to the skin and suppressing stickiness caused by application, the lower the content by percentage of the surfactant, the more preferable. For example, the content by percentage of the surfactant relative to the mass of the adenosine-containing composition is preferably 1% by mass or less.

Examples of the anionic surfactants that may be used may include fatty acid soap (such as sodium laurate, and sodium palmitate); higher alkyl sulfate ester salt (such as sodium lauryl sulfate, and potassium lauryl sulfate); alkyl ether sulfate ester salt (such as POE-lauryl sulfate triethanolamine, and sodium POE-lauryl sulfate); N-acyl sarcosinic acid (such as sodium lauroyl sarcocinate); higher fatty acid amide sulfonate (such as sodium N-stearoyl-N-methyltaurate, sodium N-myristoyl-N-methyltaurate, sodium methyl cocoyl taurate, and sodium laurylmethyl taurate); phosphate ester salt (sodium POE-oleylether phosphate, POE-stearylether phosphate, potassium cetyl phosphate); sulfosuccinate (such as sodium di-2-ethylhexyl sulfosuccinate, sodium monolauroyl monoethanolamide polyethylene sulfosuccinate, and sodium lauryl polypropylene glycol sulfosuccinate); alkylbenzene sulfonate (such as sodium linear dodecylbenzene sulfonate, triethanolamine linear dodeylbenzene sulfonate, and linear dodecylbenzene sulfonate); higher fatty acid ester sulfate ester salt (such as sodium hydrogenated gryceryl cocoate sulfate); N-acyl glutamate (such as monosodium N-lauroyl glutamate, disodium N-stearoyl glutamate, and monosodium N-myristoyl-L-glutamate); sulfonated oil (such as Turkey red oil); POE-alkyl ether carboxylic acid; POE-alkyl aryl ether carboxylate; α-olefine sulfonate; higher fatty acid ester sulfonate; secondary alcohol sulfate ester salt; higher fatty acid alkylolamide sulfate ester salt; sodium lauroyl monoethanolamide succinate; N-palmitoyl asparaginate ditriethanolamine; sodium casein; and the like.

Examples of the cationic surfactants may include alkyltrimethyl ammonium salt (such as stearyltrimethyl ammonium chloride, lauryltrimethyl ammonium chloride); alkylpyridinium salt (such as cetylpyridinium chloride); dialkyldimethyl ammonium salt (such as distearyldimethyl ammonium chloride); poly (N,N′-dimethyl-3,5-methylenepiperidinium) chloride; alkyl quaternary ammonium salt; alkyldimethylbenzyl ammonium salt; alkylisoquinolinium salt; dialkylmorphonium salt; POE alkylamine; alkylamine salt; polyamine fatty acid derivative; amyl alcohol fatty acid derivative; benzalkonium chloride; benzethonium chloride, amino acid-based cationic surfactant (such as ethyl L-cocoyl arginine DL-pyrrolidonecarboxylic acid salt) and the like.

Examples of the amphoteric surfactant that may be used may include: imidazoline-based amphoteric surfactant (such as sodium 2-undecyl-N,N,N-(hydroxyethylcarboxymethyl)-2-imidazoline and 2-cocoyl-2-imidazolinium hydroxide-1-carboxyethyloxy disodium salt); and betaine-based surfactant (such as 2-heptadecyl-N-carboxymethyl-N-hydroxyethyl imidazolinium betaine, lauryl dimethylaminoacetic acid betaine, alkyl betaine, amidobetaine, and sulfobetaine).

Examples of the hydrophilic nonionic surfactants that may be used may include POE sorbitan fatty acid ester (such as POE sorbitan monooleate, POE sorbitan monostearate, POE sorbitan monooleate, POE sorbitan tetraoleate); POE sorbit fatty acid ester (such as POE sorbit monolaurate, POE sorbit monooleate, POE sorbit pentaoleate, POE sorbit monostearate), POE glyceryl fatty acid ester (such as POE monooleate such as POE glyceryl monostearate, POE glyceryl monoisostearate, POE glyceryl triisostearate); POE fatty acid ester (such as POE distearate, POE monodioleate, ethyleneglycol distearate); POE alkyl ether (such as POE lauryl ether, POE oleyl ether, POE stearyl ether, POE behenyl ether, POE-2-octyldodecyl ether, POE cholestanol ether); puluronic type (such as Puluronic), POE/POP alkyl ethers (such as POE/POP cetyl ether, POE/POP 2-decyltetradecyl ether, POE/POP monobutyl ether, POE/POP hydrogenated lanoline, POE/POP glycerin ether); tetra POE/tetra POP ethylenediamine condensation products (such as Tetronic); POE castor oil hydrogenated castor oil derivative (such as POE caster oil, POE hydrogenated caster oil, POE hydrogenated caster oil monoisostearate, POE hydrogenated castor oil triisostearate, POE hydrogenated caster oil monopyroglutamate monoisostearate diester, POE hydrogenated oil maleate); POE beeswax/lanoline derivative (such as POE sorbitol beeswax); alkanolamide (such as coconut oil fatty acid diethanolamide, lauric acid monoethanolamide, fatty acid isopropanolamide); POE propyleneglycol fatty acid ester; POE alkyl amines; POE fatty acid amide; sucrose fatty acid ester; alkylethoxydimethylamine oxide; trioleyl phosphoric acid and the like.

Examples of the lipophilic nonionic surfactants may include sorbitan fatty acid ester (such as sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2 ethylhexylate, diglycerol sorbitan tetra-2 ethylhexylate, etc); glyceryl polyglyceryl fatty acid (such as glyceryl monocotton oil fatty acid, glyceryl monoerucate, glyceryl sesquioleate, glyceryl monostearate, glyceryl α, α′-oleate pyroglutamate, glyceryl monostearate malate, etc); propylene glycol fatty acid ester (such as propylene glycol monostearate, etc); hydrogenated caster oil derivative; glyceryl alkyl ether, and the like.

Examples of the natural water-soluble polymer may include plant-based polymer (such as gum Arabic, gum tragacanth, galactan, guar gum, locust bean gum, gum karaya, carrageenan, pectine, agar, quince seed (cydonia oblonga), algae colloid (brown algae extract), starch (rice, corn, potato, wheat),glicyrrhizic acid); microorganism based polymer (such as xanthan gum, dextran, succinoglycan, pullulan, etc), animal-based polymer (such as collagen, casein, albumin, gelatine, etc) and the like.

Examples of the semisynthetic water-soluble polymer may include starch-based polymer (such as carboxymethyl starch, methylhydroxypropyl starch, etc); cellulose-based polymer (such as methylcellulose, ethylcellulose, methylhydroxypropylcellulose, hydroxyethylcellulose, cellulose sodium sulfate, hydroxypropylcellulose, carboxymethylcellulose, sodium calboxymethyl cellulose, crystalline cellulose, cellulose powder, etc); algin acid-based polymer (such as sodium alginate, propylene glycol alginate ester, etc), and the like.

Examples of the thickeners may include gum arabic, carrageenan, karaya gum, tragacanth gum, carob gum, quince seed (marmelo), casein, dextrin, gelatin, sodium pectate, sodium alginate, methyl cellulose, ethyl cellulose, carboxymethyl cellulose (CMC), hydroxyethyl cellulose, hydroxypropyl cellulose, polyvinyl alcohol (PVA), polyvinylmethyl ether (PVM), PVP (polyvinyl pyrrolidone), polysodium acrylate, carboxyvinyl polymer, locust bean gum, guar gum, tamarind gum, dialkyldimethylammonium sulfate cellulose, xanthan gum, aluminum magnesium silicate, bentonite, hectorite, aluminum magnesium silicate (Veegum), sodium magnesium silicate (Laponite), silicic acid anhydride, taurate-based synthetic polymers, and acrylate-based synthetic polymers.

Examples of the moisturizers may include polyethylene glycol, propylene glycol, glycerin, 1,3-butylene glycol, xylitol, sorbitol, maltitol, chondroitin sulfate, hyaluronic acid, mucoitin sulfate, charonic acid, atelocollagen, cholesteryl 12-hydroxystearate, sodium lactate, bile salt, dl-pyrrolidone carboxylate, alkyleneoxide derivative, short-chain soluble collagen, diglycerin (EO)PO adduct, chestnut rose extract, yarrow extract, melilot extract, and the like.

Examples of the film-forming agent may include an anionic film-forming agent (such as (meta)acrylic acid/(meta)acrylic acid ester copolymer, methyl vinyl ether/maleic anhydride coplymer, etc), a cationic film-forming agent (such as cationic cellulose, diallyldimethylammonium chloride polymer, diallyldimethylammonium chloride/acrylic amide copolymer, etc), a nonionc film-forming agent (such as polyvinyl alcohol, polyvinylpyrrolidone, polyvinyl acetate, polyacrylic ester copolymer, (meta)acrylamide, polymeric silicone, silicone resin, trimethylsiloxysilicate, etc), and the like.

Examples of oil-soluble UV absorbers may include: benzoic acid-based UV absorbers (e.g., para-aminobenzoic acid (abbreviated as PABA hereinbelow), PABA monoglycerin ester, N,N-dipropoxy PABA ethyl ester, N,N-diethoxy PABA ethyl ester, N,N-dimethyl PABA ethyl ester, N,N-dimethyl PABA butyl ester, N,N-dimethyl PABA ethyl ester, etc.); anthranilic acid-based UV absorbers (e.g., homomenthyl-N-acetylanthranilate, etc.); salicylic acid-based UV absorbers (e.g., amyl salicylate, menthyl salicylate, homomenthyl salicylate, octyl salicylate, phenyl salicylate, benzyl salicylate, p-isopropanol phenyl salicylate, homosalate, etc.); cinnamic acid-based UV absorbers (e.g., octyl methoxycinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxycinnamate, isopropyl-p-methoxycinnamate, isoamyl-p-methoxycinnamate, octyl-p-methoxycinnamate (2-ethylhexyl-p-methoxycinnamate, ethylhexyl methoxycinnamate), 2-ethoxyethyl-p-methoxycinnamate, cyclohexyl-p-methoxycinnamate, ethyl-α-cyano-β-phenyl cinnamate, 2-ethylhexyl-α-cyano-β-phenyl cinnamate, glyceryl mono-2-ethylhexanoyl-di-para-methoxycinnamate, etc.); 3-(4′-methylbenzylidene)-d,1-camphor, 3-benzylidene-d,1-camphor; 2-phenyl-5-methylbenzoxazole; 2,2′-hydroxy-5-methylphenyl benzotriazole; 2-(2′-hydroxy-5′-t-octylphenyl)benzotriazole; 2-(2′-hydroxy-5′-methylphenyl)benzotriazole; dibenzalazine; dianisoylmethane; 4-methoxy-4′-t-butyldibenzoylmethane; 5-(3,3-dimethyl-2-norbornylidene)-3-pentan-2-one, dimorpholinopyridazinone; 2-ethylhexyl-2-cyano-3,3-diphenyl acrylate (octocrylene); 2,4-bis-{[4-(2-ethylhexyloxy)-2-hydroxy]-phenyl}-6-(4-methoxyphenyl)-(1,3,5)-triazine; and benzophenone-based UV absorbers (e.g., 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonic acid salt, 4-phenylbenzophenone, 2-ethylhexyl-4′-phenyl-benzophenone-2-carboxylate, 2-hydroxy-4-n-octoxybenzophenone, 4-hydroxy-3-carboxybenzophenone, etc.).

Examples of water-soluble UV absorbers may include: benzophenone-based UV absorbers (e.g. 2-hydroxy-4-methoxybenzophenone-5-sulfate, etc.); benzylidene camphor-based UV absorbers (e.g. benzylidene camphor sulfonic acid, terephthalylidene dicamphor sulfonic acid, etc.); phenylbenzimidazole-based UV absorbers (e.g. phenylbenzimidazole sulfonic acid, etc.)

Examples of the metal ion sequestrant may include 1-hydroxyethane-1, 1-diphosphonic acid, 1-hydroxyethane, 1-diphosphonic acid 4Na salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, trisodium hydroxyethyl ethylenediamine triacetate, and the like.

Examples of the amino acid may include neutral amino acid (such as threonine, cysteine, etc); basic amino acid (such as hydroxylysine, etc) and the like. Examples of the amino acid derivative may include sodium acyl sarcosinate (sodium lauroyl sarcosinate), acyl glutamate, sodium acyl β-alanine, glutathione, pyrrolidone carboxylate, and the like.

Examples of the organic amine may include monoethanolamine, diethanolamine, triethanolamine, morpholine, triisopropanolamine, 2-amino-2-methyl-1,3-propanediol, 2-amino-2-methyl-1-propanol, and the like.

Examples of the polymer emulsion may include acrylic resin emulsion, ethyl polyacrylate emulsion, solution of acrylic resin, polyacrylalkylester emulsion, polyvinyl acetate resin emulsion, natural rubber latex, and the like.

Examples of the pH modifier may include buffer such as lactic acid-sodium lactate, citric acid-sodium citrate, succinic acid-sodium succinate, and the like.

Examples of the vitamins may include vitamine A, B1, B2, B6, C, E and derivatives thereof, pantothenic acid and derivatives thereof, biotin, and the like.

Examples of the anti-oxidant may include tocopherols, dibutyl hydroxy toluene, butyl hydroxy anisole, and gallic acid esters, and the like.

Examples of the anti-oxidant aid may include phosphoric acid, citric acid, ascorbic acid, maleic acid, malonic acid, succinic acid, fumaric acid, cephalin, hexamethaphosphate, phytic acid, ethylenediaminetetraacetic acid, and the like.

Examples of other containable compositions may include an antiseptic agent (such as ethylparaben, butylparaben, chlorphenesin, 2-phenoxyethanol, etc); antiphlogistic (such as glycyrrhizinic acid derivatives, glycyrrhetic acid derivatives, salicylic acid derivatives, hinokitiol, zinc oxide, allantoin, etc); a skin-whitening agent (such as placental extract, saxifrage extract, arbutin, etc); various extracts (such as phellodendron bark (cork tree bark), coptis rhizome, lithospermum, peony, swertia herb, birch, sage, loquat, carrot, aloe, mallow, iris, grape, coix seed, sponge gourd, lily, saffron, cnidium rhizome, ginger, hypericum, restharrow, garlic, red pepper, citrus unshiu, Japanese angelica, seaweed, etc); an activator (such as royal jelly, photosenstizer, cholesterol derivatives, etc); a blood circulation promotion agent (such as nonylic acid vanillylamide, nicotine acid benzyl ester, nicotine acid β-butoxyethyl ester, capsaicin, zingerone, cantharides tincture, ichthammol, tannic acid, α-borneol, tocopheryl nicotinate, meso-inositol hexanicotinate, cyclandelate, cinnarizine, tolazoline, acetylcholine, verapamil, cepharanthine, γ-oryzanol, etc); an antiseborrheric agent, (such as sulfur, thianthl, etc); an anti-inflammatory agent (such as tranexamic acid, thiotaurine, hypotaurine, etc), and the like.

The composition of the present disclosure further may inculde, as necessary, caffeine, tannin, verapamil, tranexamic acid and derivatives thereof; various crude drug extracts such as licorice, Chinese quince, Pyrola japonica and the like; drugs such as tocopherol acetate, glycyrrhetinic acid, glycyrrhizic acid and derivatives thereof, or salts thereof; skin-whitening agents such as vitamin C, magnesium ascorbyl phosphate, ascorbic acid glucoside, arbutin, kojic acid and the like; amino acids such as arginine and lysine and the like and derivatives thereof.

In the adenosine-containing composition of the present disclosure, precipitation of adenosine is suppressed. For example, even when the composition is transferred from an environment at a temperature of 25° C. or higher to a low-temperature environment, the adenosine dissolved in the solvent is suppressed from precipitating out. Further, even if adenosine precipitates in a low-temperature environment, the adenosine can spontaneously dissolve simply by leaving the adenosine-containing composition to stand again in an environment at 25° C. or higher. In this way, it is possible to achieve a composition with which adenosine can be easily applied to the skin. Further, no other treatment, such as heating, for re-dissolving the precipitated adenosine is necessary. (Note that changing the environmental temperature is not encompassed within the meaning of “heating”.)

In the adenosine-containing composition of the present disclosure, ethanol for promoting dissolution of adenosine is unnecessary. Thus, the content by percentage of ethanol can be lowered, and it is even possible to contain no ethanol. In this way, the adenosine-containing composition can be made applicable to people with skin hypersensitive to ethanol and/or people allergic to ethanol.

The adenosine-containing composition of the present disclosure is applicable to external-use skin preparations, particularly external-use skin preparations applicable to the scalp. The adenosine-containing composition of the present disclosure is applicable, for example, to hair restorers, hair tonics, hair increasing agents, hair growth agents, hair loss inhibitors, shampoos, hair treatments, conditioners, etc.

A method for manufacturing the adenosine-containing composition of the present disclosure will be described. For example, a precipitation-suppressing component is dissolved in an aqueous solvent including water. Next, adenosine is added to and dissolved in the aqueous solvent. In this way, the adenosine-containing composition of the present disclosure can be prepared. The order of addition is not limited, so long as each of the components can be dissolved. It is preferred to dissolve adenosine in an aqueous solvent having a temperature of 25° C. or higher.

There may be cases where it is difficult, or utterly impractical, to directly define the adenosine-containing composition of the present disclosure based on the compositional makeup, structure, etc., thereof. In such circumstances, it should be permissible to define the adenosine-containing composition of the present disclosure according to methods for producing the same.

A method for suppressing precipitation of adenosine according to a second embodiment of the present disclosure will be described. This method includes a dissolution step of dissolving adenosine and a first component to an aqueous solvent. The first component is at least one selected from the group consisting of caffeine, glycyrrhizic acid and salts thereof, salicylic acid and salts thereof, and plant extracts. The first component corresponds to the precipitation-suppressing component as referred to in the first embodiment. The order for adding each of the components is not limited.

In the dissolution step, it is preferred to dissolve adenosine in an aqueous solvent having a temperature of 25° C. or higher.

In cases where, in the dissolution step, the first component does not dissolve into the aqueous solvent, the method for suppressing precipitation of adenosine may further include a step of adding a second component, such as a surfactant, etc., capable of making the first component dissolvable into the aqueous solvent.

The method for suppressing precipitation of adenosine may further include a step of placing (storing) the composition, which has been prepared in the dissolution step, in an environment at room temperature, i.e., 25° C., or higher. In this way, precipitation of adenosine can be suppressed. Even if adenosine precipitates out due to a drop in solubility at low temperatures, it is possible to promote spontaneous dissolution of adenosine.

The amount of adenosine added may be the same as that in the first embodiment. As regards the amount of adenosine to be added, the description of the first embodiment is incorporated herein by reference, and description thereon is omitted herefrom. It is preferred that adenosine is dissolved in an aqueous solvent at 25° C. or higher.

The types of precipitation-suppressing components and the amount to be added may be the same as in the first embodiment. As regards the types of precipitation-suppressing components and the amount to be added, the description of the first embodiment is incorporated herein by reference, and description thereon is omitted herefrom.

The aqueous solvent includes water. The content of water relative to the mass of the aqueous solvent may be 60% by mass or greater. The amount of water added may be the same as that in the first embodiment. As regards the amount of water to be added, the description of the first embodiment is incorporated herein by reference, and description thereon is omitted herefrom.

The content of ethanol relative to the total mass of the aqueous solvent, or the various components, is less than 20% by mass, preferably 15% by mass or less, more preferably 10% by mass or less, more preferably 5% by mass or less, more preferably 3% by mass or less, more preferably 2% by mass or less, more preferably 1% by mass or less, and it is even more preferred that substantially no ethanol (0% by mass) is included.

It is preferred that the method for suppressing precipitation of adenosine does not include a step of adding ethanol.

The method for suppressing precipitation of adenosine may further include a step of adding a polyol to the aqueous solvent and/ or to the composition prepared in the dissolution step. The types of polyols and the amount to be added may be the same as in the first embodiment. As regards the types of polyols and the amount to be added, the description of the first embodiment is incorporated herein by reference, and description thereon is omitted herefrom.

It is preferred that the composition prepared in the method for suppressing precipitation of adenosine is a single-phase liquid composition in which component (A) and component (B) have been dissolved.

According to the method for suppressing precipitation of adenosine, it is possible to suppress precipitation of adenosine dissolved in an aqueous solvent, even if the amount of ethanol used is small. Particularly, it is possible to suppress precipitation of adenosine in a low-temperature environment. Further, even if adenosine precipitates out in a low-temperature environment, the precipitate can spontaneously dissolve by being returned to an environment of 25° C. or higher, and adenosine can be maintained in a dissolved state at room temperature. Furthermore, treatment, such as heating, for re-dissolving the precipitated adenosine is unnecessary.

### EXAMPLES

Adenosine-containing compositions and adenosine precipitation suppressing methods of the present disclosure will be described below by way of examples. Note, however, that the adenosine-containing compositions and adenosine precipitation suppressing methods of the present disclosure are not limited to the following examples. The content by percentage of each of the components shown in the Tables is in terms of percent by mass (mass%).

### Test Examples 1 to 98

Adenosine-containing compositions were prepared at 25° C. The adenosine-containing compositions immediately after preparation were single-phase aqueous compositions, and the adenosine and the precipitation-suppressing components were dissolved in the respective compositions. The prepared adenosine-containing compositions were left to stand in a 0° C. environment for 4 weeks, and then whether precipitation occurred in the compositions or not was visually observed. Next, the adenosine-containing compositions left to stand in a 0° C. environment for 4 weeks were then left to stand in a 25° C. environment for 7 days, and then whether any precipitate remained in each composition or not (i.e., whether the precipitate re-dissolved or not) was visually observed. The test results were evaluated according to the following criteria. The plant extracts were commercially available products.

A: No precipitate was produced even in a 0° C. environment.

B: Precipitate was produced in a 0° C. environment, but the precipitate spontaneously re-dissolved when the composition was returned to a 25° C. environment.

C: Precipitate was produced in a 0° C. environment, and the precipitate remained even after the composition was returned to a 25° C. environment.

### Test Examples 1 to 36

Using the composition shown in Table 1 as the basic composition, the types of precipitation-suppressing components (B) were varied, to perform precipitation tests. Table 2 shows the tested precipitation-suppressing components, their content by percentage (X% by mass), and test results.

In Test Example 36 which did not contain ethanol nor any precipitation-suppressing component, the adenosine which precipitated at 0° C. did not re-dissolve even when returned to 25° C. In contrast, in Test Examples 1 to 35 in which precipitation-suppressing components were added, either no adenosine precipitated at 0° C., or even if it did, the adenosine re-dissolved spontaneously when returned to 25° C. From these results, it is thought that the precipitation-suppressing components have an action of suppressing precipitation of adenosine or an action of facilitating re-dissolution of precipitated adenosine (or an action of causing easily re-dissolvable adenosine to precipitate).

TABLE 1

| | |
|---|---|
| Test Example | 1-36 |
| (A) Adenosine | 0.5 |
| (B) Precipitation-suppressing component | X |
| (C) Ion-exchanged water | balance |
| (D) Dipropylene glycol | 10 |
| (D) 1,3-Butylene glycol | 10 |
| POE/POP decyltetradecyl ether | 0.03 |
| Phenoxyethanol | q.s. |
| Citric acid | q.s. |
| Sodium citrate | q.s. |
| Total | 100 |

TABLE 2

| Test Example No. | (B) Precipitation-suppressing component | Addition rate X (% by mass) | Result |
|---|---|---|---|
| 1 | Caffeine | 0.05 | B |
| 2 | Dipotassium glycyrrhizinate | 0.1 | A |
| 3 | Monoammonium glycyrrhizinate | 0.1 | A |
| 4 | Sodium salicylate | 0.1 | A |
| 5 | Angelica keiskei leaf/stem extract | 0.005 | B |
| 6 | Prunus armeniaca (apricot) juice | 0.2 | A |
| 7 | Ginkgo biloba leaf extract | 0.01 | A |
| 8 | Citrus unshiu peel extract | 0.01 | B |
| 9 | Scutellaria baicalensis root extract | 0.01 | B |
| 10 | Panax ginseng root extract | 0.005 | B |
| 11 | Rosa canina fruit extract | 0.005 | B |
| 12 | Ononis spinosa extract | 0.005 | B |
| 13 | Glycyrrhiza glabra (licorice) root extract | 0.015 | A |
| 14 | Phellodendron amurense bark extract | 0.003 | B |
| 15 | Sophora angustifolia root extract | 0.015 | A |
| 16 | Prunus speciosa leaf extract | 0.01 | B |
| 17 | Hypericum perforatum flower/leaf/stem extract | 0.005 | B |
| 18 | Crataegus monogyna flower extract | 0.02 | B |
| 19 | Aesculus hippocastanum (horse chestnut) seed extract | 0.002 | B |
| 20 | Achillea millefolium extract | 0.001 | B |
| 21 | Rosa centifolia flower extract | 0.005 | B |
| 22 | Prunus yedoensis leaf extract | 0.01 | B |
| 23 | Camellia japonica seed extract | 0.003 | B |
| 24 | Camellia japonica flower extract | 0.004 | A |
| 25 | Rubus suavissimus (raspberry) extract | 0.004 | B |
| 26 | Nelumbo nucifera germ extract | 0.004 | B |
| 27 | Syzygium jambos leaf extract | 0.004 | B |
| 28 | Bupleurum falcatum root extract | 0.001 | B |
| 29 | Melilotus officinalis extract | 0.004 | B |
| 30 | Centaurea cyanus flower extract | 0.005 | B |
| 31 | Citrus limon (lemon) extract | 0.2 | B |
| 32 | Rosmarinus officinalis (rosemary) extract | 0.001 | B |
| 33 | Rosmarinus officinalis (rosemary) leaf extract | 0.006 | B |
| 34 | Thymus serpyllum extract | 0.005 | A |
| 35 | Sanguisorba officinalis extract | 0.004 | A |
| 36 | (Not added) | - | C |

### Test Examples 37 to 72

Using some of the precipitation-suppressing components used in Test Examples 1 to 35, compositions were prepared by changing the content by percentage of the precipitation-suppressing components or by employing a plurality of precipitation-suppressing components in combination. In some Test Examples, the amount of adenosine was increased compared to Test Examples 1 to 36. A surfactant was added in cases where the precipitation-suppressing component and perfume did not dissolve into the solvent. All of the compositions of Test Examples 37 to 72 were a single-phase aqueous composition immediately after preparation, and adenosine and the precipitation-suppressing components were dissolved in the solvent. Tables 3 to 8 show the respective compositions and test results.

In all of Test Examples 37 to 49 in which the content by percentage of the precipitation-suppressing components was changed from that in Test Examples 1 to 35, it was possible to confirm a precipitation-suppressing effect. It is thus thought that the content of the precipitation-suppressing component can be 1×10⁻⁴% by mass or greater. With consideration given also to the results of Test Examples 1 to 35, a tendency was observed for the precipitation-suppressing effect to improve with an increase in the amount of precipitation-suppressing component with respect to adenosine.

Also in Test Examples 50 to 72 in which a plurality of precipitation-suppressing components were used in combination, it was possible to confirm a precipitation-suppressing effect.

TABLE 3

| | | | | |
|---|---|---|---|---|
| Test Example | 37 | 38 | 39 | 40 |
| (A) Adenosine | 1 | 1 | 1 | 1 |
| (B) Dipotassium glycyrrhizinate | - | - | - | - |
| (B) Monoammonium glycyrrhizinate | 0.1 | 0.2 | 0.2 | - |
| (B) Caffeine | - | - | - | 0.2 |
| (C) Ion-exchanged water | balance | balance | balance | balance |
| (D) Dipropylene glycol | 20 | 20 | 10 | 20 |
| (D) 1,3-Butylene glycol | - | - | - | - |
| POE/POP decyltetradecyl ether | - | - | - | - |
| Phenoxyethanol | q.s. | q.s. | q.s. | q.s. |
| Citric acid | q.s. | q.s. | q.s. | q.s. |
| Sodium citrate | q.s. | q.s. | q.s. | q.s. |
| Total | 100 | 100 | 100 | 100 |
| Result | B | A | B | A |

TABLE 4

| | | | | |
|---|---|---|---|---|
| Test Example | 41 | 42 | 43 | 44 |
| (A) Adenosine | 1 | 1 | 1 | 1 |
| (B) Prunus armeniaca (apricot) juice | 0.85 | - | - | - |
| (B) Glycyrrhiza glabra (licorice) root extract | - | 0.05 | - | - |
| (B) Sophora angustifolia root extract | - | - | 0.004 | 0.02 |
| (C) Ion-exchanged water | balance | balance | balance | balance |
| (D) Dipropylene glycol | 20 | 20 | 20 | 20 |
| (D) 1,3-Butylene glycol | - | - | - | - |
| POE/POP decyltetradecyl ether | 0.05 | 0.05 | 0.03 | 0.05 |
| Phenoxyethanol | q.s. | q.s. | q.s. | q.s. |
| Citric acid | q.s. | q.s. | q.s. | q.s. |
| Sodium citrate | q.s. | q.s. | q.s. | q.s. |
| Total | 100 | 100 | 100 | 100 |
| Result | A | B | B | A |

TABLE 5

| | | | | | |
|---|---|---|---|---|---|
| Test Example | 45 | 46 | 47 | 48 | 49 |
| (A) Adenosine | 1 | 0.5 | 0.2 | 0.5 | 1 |
| (B) Crataegus monogyna flower extract | 0.04 | - | - | - | - |
| (B) Aesculus hippocastanum (horse | - | 0.4 | - | - | - |
| chestnut) seed extract | - | - | - | - | - |
| (B) Achillea millefolium extract | - | - | 0.0003 | 0.005 | - |
| (B) Citrus limon (lemon) extract | - | - | - | - | 0.6 |
| (C) Ion-exchanged water | balance | balance | balance | balance | balance |
| (D) Dipropylene glycol | 20 | 10 | 20 | 10 | 20 |
| (D) 1,3-Butylene glycol | - | 10 | - | 10 | - |
| POE/POP decyltetradecyl ether | 0.05 | 0.05 | 0.02 | 0.03 | 0.05 |
| Phenoxyethanol | q.s. | q.s. | q.s. | q.s. | q.s. |
| Citric acid | q.s. | q.s. | q.s. | q.s. | q.s. |
| Sodium citrate | q.s. | q.s. | q.s. | q.s. | q.s. |
| Total | 100 | 100 | 100 | 100 | 100 |
| Result | B | B | B | B | A |

TABLE 6

| | | | | | | | |
|---|---|---|---|---|---|---|---|
| Test Example | 50 | 51 | 52 | 53 | 54 | 55 | 56 |
| (A) Adenosine | 1 | 1 | 1 | 1 | 1 | 1 | 1 |
| (B) Dipotassium glycyrrhizinate | 0.1 | 0.1 | 0.1 | 0.1 | 0.1 | - | - |
| (B) Monoammoniu m | 0.1 | - | - | - | - | 0.1 | 0.1 |
| glycyrrhizinate (B) Sodium salicylate | - | - | - | - | 0.1 | - | - |
| (B) Caffeine | - | - | - | 0.1 | - | - | - |
| (B) Sophora angustifolia root extract | - | 0.004 | 0.02 | - | - | 0.004 | 0.02 |
| (C) Ion-exchanged water | balance | balance | balance | balance | balance | balance | balance |
| (D) Dipropylene glycol | 20 | 20 | 20 | 20 | 20 | 20 | 20 |
| (D) 1,3-Butylene glycol | - | - | - | - | - | - | - |
| POE/POP decyltetradecyl ether | - | 0.03 | 0.05 | - | - | 0.03 | 0.05 |
| Phenoxyethanol | q.s. | q.s. | q.s. | q.s. | q.s. | q.s. | q.s. |
| Citric acid | q.s. | q.s. | q.s. | q.s. | q.s. | q.s. | q.s. |
| Sodium citrate | q.s. | q.s. | q.s. | q.s. | q.s. | q.s. | q.s. |
| Total | 100 | 100 | 100 | 100 | 100 | 100 | 100 |
| Result | B | B | A | B | B | A | A |

TABLE 7

| | | | | | | | |
|---|---|---|---|---|---|---|---|
| Test Example | 57 | 58 | 59 | 60 | 61 | 62 | 63 |
| (A) Adenosine | 1 | 1 | 1 | 1 | 1 | 1 | 1 |
| (B) Dipotassium glycyrrhizinate | - | - | - | - | - | - | - |
| (B) Monoammoniu m | 0.1 | 0.1 | - | - | - | - | - |
| glycyrrhizinate (B) Sodium salicylate | - | 0.1 | - | - | 0.1 | 0.1 | 0.1 |
| (B) Caffeine | 0.1 | - | 0.1 | 0.1 | - | - | 0.1 |
| (B) Sophora angustifolia root extract | - | - | 0.004 | 0.02 | 0.004 | 0.02 | - |
| (C) Ion-exchanged water | balance | balance | balance | balance | balance | balance | balance |
| (D) Dipropylene glycol | 20 | 20 | 20 | 20 | 20 | 20 | 20 |
| (D) 1,3-Butylene glycol | - | - | - | - | - | - | - |
| POE/POP decyltetradecyl ether | - | - | 0.03 | 0.05 | 0.03 | 0.05 | - |
| Phenoxyethanol | q.s. | q.s. | q.s. | q.s. | q.s. | q.s. | q.s. |
| Citric acid | q.s. | q.s. | q.s. | q.s. | q.s. | q.s. | q.s. |
| Sodium citrate | q.s. | q.s. | q.s. | q.s. | q.s. | q.s. | q.s. |
| Total | 100 | 100 | 100 | 100 | 100 | 100 | 100 |
| Result | B | B | B | A | B | A | B |

TABLE 8

| | | | | | | | | | |
|---|---|---|---|---|---|---|---|---|---|
| Test Example | 64 | 65 | 66 | 67 | 68 | 69 | 70 | 71 | 72 |
| (A) Adenosine | 1 | 0.5 | 1 | 1 | 1 | 0.5 | 0.5 | 1 | 1 |
| (B) Dipotassium glycyrrhizin ate | - | - | 0.1 | - | - | - | - | 0.1 | 0.1 |
| (B) Monoammo nium glycyrrhizin ate | 0.1 | 0.1 | - | 0.1 | 0.1 | 0.1 | - | - | - |
| (B) Sodium salicylate | 0.1 | 0.1 | 0.1 | 0.1 | - | - | - | - | 0.1 |
| (B) Caffeine | - | - | - | - | - | - | - | - | - |
| (B) Sophora angustifolia root extract | 0.02 | 0.02 | 0.02 | - | 0.02 | 0.02 | 0.02 | 0.02 | - |
| (C) Ion-exchanged water | balance | balance | balance | balance | balance | balance | balance | balance | balance |
| (D) Dipropylene glycol | 10 | 10 | 10 | 10 | 10 | 10 | 10 | 10 | 10 |
| (D) 1,3-Butylene glycol | 10 | 10 | 10 | 10 | 10 | 10 | 10 | 10 | 10 |
| POE/POP decyltetrade cyl ether | 0.05 | 0.5 | 0.05 | - | 0.05 | 0.5 | 0.05 | 0.05 | - |
| Nicotinamid e | 0.1 | 0.1 | 0.1 | 0.1 | 0.1 | 0.1 | 0.1 | 0.1 | 0.1 |
| Phenoxyeth anol | q.s. | q.s. | q.s. | q.s. | q.s. | q.s. | q.s. | q.s. | q.s. |
| Citric acid | q.s. | q.s. | q.s. | q.s. | q.s. | q.s. | q.s. | q.s. | q.s. |
| Sodium citrate | q.s. | q.s. | q.s. | q.s. | q.s. | q.s. | q.s. | q.s. | q.s. |
| Perfume | - | q.s. | - | - | - | q.s. | - | - | - |
| Total | 100 | 100 | 100 | 100 | 100 | 100 | 100 | 100 | 100 |
| Result | A | A | A | B | A | A | A | A | B |

### Test Examples 73 to 81

In Test Examples 73 to 78, compositions containing ethanol but not containing any precipitation-suppressing component were prepared. Note, however, that the presence/absence of precipitation was confirmed after each composition was cooled and then left to stand at room temperature for 4 weeks. In Test Example 79, a composition containing a polyol but not containing ethanol nor any precipitation-suppressing component was prepared. In Test Example 80, a composition containing a precipitation-suppressing component but not containing ethanol nor polyol was prepared. In Test Example 81, a composition containing a precipitation-suppressing component, ethanol, and a polyol was prepared. All of the compositions of Test Examples 73 to 81 were a single-phase aqueous composition immediately after preparation, and adenosine and the precipitation-suppressing components were dissolved in the solvent. Tables 9 and 10 show the respective compositions and test results.

Test Examples 76 to 78, which had a high content by percentage of ethanol, had a high adenosine precipitation-suppressing effect. In contrast, Test Examples 73 to 75, which had a low content by percentage of ethanol, had no adenosine precipitation-suppressing effect. It is thus thought that compositions containing no precipitation-suppressing component cannot sufficiently suppress precipitation when the content of ethanol is less than 20% by mass.

Test Example 79, which contained only a polyol, also could not achieve a sufficient adenosine precipitation-suppressing effect. Test Example 80 showed that a precipitation-suppressing effect can be obtained by the precipitation-suppressing component, even without containing ethanol or polyol. Test Example 81 showed that a precipitation-suppressing effect can be obtained by the precipitation-suppressing component, even in the presence of ethanol.

TABLE 9

| | | | | | | |
|---|---|---|---|---|---|---|
| Test Example | 73 | 74 | 75 | 76 | 77 | 78 |
| (A) Adenosine | 1 | 1 | 1 | 1 | 1 | 1 |
| (B) Caffeine | - | - | - | - | - | - |
| (C) Ion-exchanged water | balance | balance | balance | balance | balance | balance |
| Ethanol | 5 | 10 | 15 | 40 | 40 | 50 |
| Dipropylene glycol | - | - | - | 10 | - | - |
| Phenoxyethanol | - | - | - | - | - | - |
| Citric acid | q.s. | q.s. | q.s. | - | - | - |
| Sodium citrate | q.s. | q.s. | q.s. | - | - | - |
| Total | 100 | 100 | 100 | 100 | 100 | 100 |
| Result | C | C | C | A | B | A |

TABLE 10

| | | | |
|---|---|---|---|
| Test Example | 79 | 80 | 81 |
| (A) Adenosine | 1 | 0.5 | 0.5 |
| (B) Caffeine | - | 0.2 | 0.2 |
| (C) Ion-exchanged water | balance | balance | balance |
| Ethanol | - | - | 10 |
| Dipropylene glycol | 20 | - | 10 |
| Phenoxyethanol | q.s. | - | q.s. |
| Citric acid | q.s. | q.s. | q.s. |
| Sodium citrate | q.s. | q.s. | q.s. |
| Total | 100 | 100 | 100 |
| Result | C | B | A |

### Test Examples 82 to 85

Compositions were prepared by changing the content by percentage of adenosine. A surfactant was added in cases where the precipitation-suppressing component did not dissolve into the solvent. All of the compositions of Test Examples 82 to 85 were a single-phase aqueous composition immediately after preparation, and adenosine and the precipitation-suppressing components were dissolved in the solvent. Tables 11 shows the respective compositions and test results.

In all of Test Examples 82 to 85, it was possible to confirm a precipitation-suppressing effect. This shows that the content of adenosine relative to the mass of the composition can be 5×10⁻⁴% by mass or greater, preferably 8×10⁻⁴% by mass or greater. This also shows that the content of adenosine relative to the mass of the composition can be 8% by mass or less, preferably 6% by mass or less.

TABLE 11

| | | | | |
|---|---|---|---|---|
| Test Example | 82 | 83 | 84 | 85 |
| (A) Adenosine | 0.001 | 0.2 | 2 | 5 |
| (B) Sophora angustifolia root extract | 0.02 | 0.02 | 0.02 | 0.02 |
| (C) Ion-exchanged water | balance | balance | balance | balance |
| (D) Dipropylene glycol | 20 | 20 | 20 | 20 |
| POE/POP decyltetradecyl ether | 0.05 | 0.05 | 0.05 | 0.05 |
| Phenoxyethanol | q.s. | q.s. | q.s. | q.s. |
| Citric acid | q.s. | q.s. | q.s. | q.s. |
| Sodium citrate | q.s. | q.s. | q.s. | q.s. |
| Total | 100 | 100 | 100 | 100 |
| Result | A | A | A | B |

### Test Examples 86 to 89

Compositions were prepared by changing the combination and content by percentage of the plant extracts. A surfactant was added in cases where the precipitation-suppressing component did not dissolve into the solvent. All of the compositions of Test Examples 86 to 89 were a single-phase aqueous composition immediately after preparation, and adenosine and the precipitation-suppressing components were dissolved in the solvent. Tables 12 shows the respective compositions and test results.

In all of Test Examples 86 to 89, it was possible to confirm a precipitation-suppressing effect. It is thus thought that a precipitation-suppressing effect can be obtained even when plant extracts, each confirmed in the aforementioned Test Examples as having an adenosine precipitation-suppressing effect by itself, are employed in combination. This also shows that the content of the plant extract relative to the mass of the composition can be 0.5% by mass or greater.

TABLE 12

| | | | | |
|---|---|---|---|---|
| Test Example | 86 | 87 | 88 | 89 |
| (A) Adenosine | 0.2 | 1 | 1 | 1 |
| (B) Prunus armeniaca (apricot) juice | - | - | 0.85 | - |
| (B) Ginkgo biloba leaf extract | - | - | - | 0.02 |
| (B) Sophora angustifolia root extract | - | 0.02 | 0.02 | 0.02 |
| (B) Citrus limon (lemon) extract | 0.6 | 0.6 | - | - |
| (C) Ion-exchanged water | balance | balance | balance | balance |
| (D) Dipropylene glycol | 20 | 20 | 20 | 20 |
| POE/POP decyltetradecyl ether | 0.05 | 0.05 | 0.05 | 0.05 |
| Phenoxyethanol | q.s. | q.s. | q.s. | q.s. |
| Citric acid | q.s. | q.s. | q.s. | q.s. |
| Sodium citrate | q.s. | q.s. | q.s. | q.s. |
| Total | 100 | 100 | 100 | 100 |
| Result | A | A | A | A |

### Test Examples 90 to 98

Compositions were prepared by using a plurality of precipitation-suppressing components in combination. A surfactant was added in cases where the precipitation-suppressing component did not dissolve into the solvent. All of the compositions of Test Examples 90 to 98 were a single-phase aqueous composition immediately after preparation, and adenosine and the precipitation-suppressing components were dissolved in the solvent. Tables 13 shows the respective compositions and test results.

In Test Examples 90 to 98 in which a plurality of precipitation-suppressing components were used in combination, it was possible to confirm a precipitation-suppressing effect. This shows that a precipitation-suppressing effect can be obtained even when components, each having an adenosine precipitation-suppressing effect by itself, are employed in combination.

TABLE 13

| | | | | | | | | | |
|---|---|---|---|---|---|---|---|---|---|
| Test Example | 90 | 91 | 92 | 93 | 94 | 95 | 96 | 97 | 98 |
| (A) Adenosine | 1 | 1 | 0.2 | 1 | 1 | 1 | 1 | 1 | 1 |
| (B) Glycyrrhizic acid | - | 0.1 | - | - | - | - | - | - | - |
| (B) Dipotassium glycyrrhizin ate | - | - | - | - | - | 0.1 | - | - | - |
| (B) Monoammo nium glycyrrhizin ate | - | - | 0.1 | - | 0.1 | - | 0.1 | 0.1 | 0.1 |
| (B) Sodium salicylate | 0.1 | - | - | - | - | - | - | - | - |
| (B) Sodium salicylate | - | - | - | 0.1 | - | - | - | 0.1 | - |
| (B) Caffeine | - | | 0.1 | 0.1 | 0.1 | 0.1 | 0.1 | - | 0.1 |
| (B) Sophora angustifolia root extract | 0.02 | 0.02 | 0.02 | 0.02 | 0.02 | 0.02 | - | 0.02 | 0.02 |
| (B) Citrus limon (lemon) extract | - | - | - | - | - | - | 0.6 | - | - |
| (C) Ion-exchanged water | balance | balance | balance | balance | balance | balance | balance | balance | balance |
| (D) Dipropylene glycol | 20 | 20 | 20 | 20 | 20 | 20 | 20 | 20 | 10 |
| (D) 1,3-Butylene glycol | - | - | - | - | - | - | - | - | 10 |
| POE/POP decyltetrade cyl ether | 0.05 | 0.5 | 0.05 | 0.05 | 0.05 | 0.5 | 0.05 | 0.05 | 0.05 |
| Ethanol | - | - | - | - | - | - | - | - | 5 |
| Phenoxyeth anol | q.s. | q.s. | q.s. | q.s. | q.s. | q.s. | q.s. | q.s. | q.s. |
| Citric acid | q.s. | q.s. | q.s. | q.s. | q.s. | q.s. | q.s. | q.s. | q.s. |
| Sodium citrate | q.s. | q.s. | q.s. | q.s. | q.s. | q.s. | q.s. | q.s. | q.s. |
| Total | 100 | 100 | 100 | 100 | 100 | 100 | 100 | 100 | 100 |
| Result | B | B | A | A | A | A | A | A | A |

The composition containing adenosine and the method for suppressing the precipitation of adenosine according to the present invention have been described according to the foregoing embodiments and examples, but the invention is not limited to the foregoing embodiments and examples and may encompass various transformations, modifications, and improvements made to the various disclosed elements (including elements disclosed in the Claims, Description, and Drawings) within the scope of the invention and according to the fundamental technical idea of the present invention. Further, various combinations, substitutions, and selections of the various disclosed elements are possible within the scope of the claims of the invention.

Further issues, objectives, and embodiments (including modifications) of the present invention are revealed also from the entire disclosure of the invention including the Claims.

The numerical ranges disclosed herein are to be construed in such a manner that arbitrary numerical values and ranges falling within the disclosed ranges are treated as being concretely described herein, even where not specifically stated.

Some or all of the foregoing embodiments may be described as in the following additional features, although not limited thereto. The various additional features may be employed in combination with the claim(s) in the Scope of Claims.

### Additional Feature 1

An adenosine-containing composition comprising:
(A) adenosine;
(B) at least one selected from the group consisting of caffeine, glycyrrhizic acid and salts thereof, salicylic acid and salts thereof, and plant extracts; and
(C) water, wherein
the content of ethanol is less than 20% by mass relative to the mass of the adenosine-containing composition.

### Additional Feature 2

The adenosine-containing composition according to Additional Feature (s), wherein the plant extract is at least one selected from the group consisting of Angelica keiskei leaf/stem extract, Prunus armeniaca (apricot) juice, Ginkgo biloba leaf extract, Citrus unshiu peel extract, Scutellaria baicalensis root extract, Panax ginseng root extract, Rosa canina fruit extract, Ononis spinosa extract, Glycyrrhiza glabra (licorice) root extract, Phellodendron amurense bark extract, Sophora angustifolia root extract, Prunus speciosa leaf extract, Hypericum perforatum flower/leaf/stem extract, Crataegus monogyna flower extract, Aesculus hippocastanum (horse chestnut) seed extract, Achillea millefolium extract, Rosa centifolia flower extract, Prunus yedoensis leaf extract, Camellia japonica seed extract, Camellia japonica flower extract, Rubus suavissimus (raspberry) extract, Nelumbo nucifera germ extract, Syzygium jambos leaf extract, Bupleurum falcatum root extract, Melilotus officinalis extract, Centaurea cyanus flower extract, Citrus limon (lemon) extract, Rosmarinus officinalis (rosemary) extract, Rosmarinus officinalis (rosemary) leaf extract, Thymus serpyllum extract, and Sanguisorba officinalis extract.

### Additional Feature 3

The adenosine-containing composition according to Additional Feature (s), wherein a content by percentage of the component (A) relative to the mass of the adenosine-containing composition is from 5×10⁻⁴% by mass to 8% by mass.

### Additional Feature 4

The adenosine-containing composition according to Additional Feature (s), wherein the component (B) is a component which suppresses precipitation of the dissolved component (A).

### Additional Feature 5

The adenosine-containing composition according to Additional Feature (s), wherein a content by percentage of the component (B) relative to the mass of the adenosine-containing composition is 1×10⁻⁴% by mass or greater.

### Additional Feature 6

The adenosine-containing composition according to Additional Feature (s), wherein a content by percentage of the component (C) relative to the mass of the adenosine-containing composition is 60% by mass or greater.

### Additional Feature 7

The adenosine-containing composition according to any one of Additional Feature (s), further comprising from 5% by mass to 40% by mass of a polyol relative to the mass of the adenosine-containing composition.

### Additional Feature 8

The adenosine-containing composition according to Additional Feature (s), wherein:
the composition is a single-phase liquid composition; and
in atmospheric pressure at 25° C., the component (A) and the component (B) are dissolved in the adenosine-containing composition.

### Additional Feature 9

The adenosine-containing composition according to Additional Feature (s), comprising substantially no ethanol.

### Additional Feature 10

The adenosine-containing composition according to Additional Feature (s), wherein the composition is applicable in an external-use skin preparation applicable to the skin.

### Additional Feature 11

The adenosine-containing composition according to Additional Feature (s), wherein the composition is an external-use skin preparation for application to the scalp.

### Additional Feature 12

A method for suppressing precipitation of adenosine, comprising:
dissolving adenosine and a first component to an aqueous solvent containing less than 20% by mass of ethanol, wherein
the first component is at least one selected from the group consisting of caffeine, glycyrrhizic acid and salts thereof, salicylic acid and salts thereof, and plant extracts.

### Additional Feature 13

The method according to Additional Feature (s), wherein, in the aqueous solvent, a content of water relative to the mass of the aqueous solvent is 60% by mass or greater.

### Additional Feature 14

The method according to Additional Feature (s), wherein the plant extract is at least one selected from the group consisting of Angelica keiskei leaf/stem extract, Prunus armeniaca (apricot) juice, Ginkgo biloba leaf extract, Citrus unshiu peel extract, Scutellaria baicalensis root extract, Panax ginseng root extract, Rosa canina fruit extract, Ononis spinosa extract, Glycyrrhiza glabra (licorice) root extract, Phellodendron amurense bark extract, Sophora angustifolia root extract, Prunus speciosa leaf extract, Hypericum perforatum flower/leaf/stem extract, Crataegus monogyna flower extract, Aesculus hippocastanum (horse chestnut) seed extract, Achillea millefolium extract, Rosa centifolia flower extract, Prunus yedoensis leaf extract, Camellia japonica seed extract, Camellia japonica flower extract, Rubus suavissimus (raspberry) extract, Nelumbo nucifera germ extract, Syzygium jambos leaf extract, Bupleurum falcatum root extract, Melilotus officinalis extract, Centaurea cyanus flower extract, Citrus limon (lemon) extract, Rosmarinus officinalis (rosemary) extract, Rosmarinus officinalis (rosemary) leaf extract, Thymus serpyllum extract, and Sanguisorba officinalis extract.

### Additional Feature 15

The method according to Additional Feature (s), wherein from 5×10⁻⁴% by mass to 8% by mass of adenosine is added to a total mass of composition.

### Additional Feature 16

The method according to Additional Feature (s), wherein the first component is a component which suppresses precipitation of the dissolved adenosine.

### Additional Feature 17

The method according to Additional Feature (s), wherein 1×10⁻⁴% by mass or greater of the first component is added to the total mass of the composition.

### Additional Feature 18

The method according to Additional Feature (s), further comprising adding, to the aqueous solvent, from 5% by mass to 40% by mass of a polyol relative to the total mass of the composition.

### Additional Feature 19

The method according to Additional Feature (s), wherein a single-phase liquid composition in which component (A) and component (B) have been dissolved is prepared.

### Additional Feature 20

The method according to Additional Feature (s), not comprising adding ethanol.

### Additional Feature 21

A method of using an adenosine-containing composition, comprising using the adenosine-containing composition of the present disclosure as an external-use skin preparation.

### Additional Feature 22

A method of using an adenosine-containing composition, comprising applying the adenosine-containing composition of the present disclosure to the scalp.

### INDUSTRIAL APPLICABILITY

The adenosine-containing composition and adenosine precipitation suppressing method of the present disclosure are applicable, for example, to external-use skin preparations, cleansers, etc. The adenosine-containing composition and the adenosine precipitation suppressing method are applicable, for example, to external-use skin preparations and cleansers applicable to the scalp and the hair. Examples of applicable external-use skin preparations and cleansers may include hair restorers, hair tonics, hair increasing agents, hair growth agents, hair loss inhibitors, shampoos, hair treatments, conditioners, etc.

1. An adenosine-containing composition comprising:
(A) adenosine;
(B) at least one selected from the group consisting of salicylic acid and salts thereof, glycyrrhizic acid and salts thereof, and plant extracts; and
(C) water, wherein
a content of ethanol is less than 20% by mass relative to the mass of the adenosine-containing composition.

2. The adenosine-containing composition according to claim 1, wherein the plant extract is at least one selected from the group consisting of Angelica keiskei leaf/stem extract, Prunus armeniaca (apricot) juice, Ginkgo biloba leaf extract, Citrus unshiu peel extract, Scutellaria baicalensis root extract, Panax ginseng root extract, Rosa canina fruit extract, Ononis spinosa extract, Glycyrrhiza glabra (licorice) root extract, Phellodendron amurense bark extract, Sophora angustifolia root extract, Prunus speciosa leaf extract, Hypericum perforatum flower/leaf/stem extract, Crataegus monogyna flower extract, Aesculus hippocastanum (horse chestnut) seed extract, Achillea millefolium extract, Rosa centifolia flower extract, Prunus yedoensis leaf extract, Camellia japonica seed extract, Camellia japonica flower extract, Rubus suavissimus (raspberry) extract, Nelumbo nucifera germ extract, Syzygium jambos leaf extract, Bupleurum falcatum root extract, Melilotus officinalis extract, Centaurea cyanus flower extract, Citrus limon (lemon) extract, Rosmarinus officinalis (rosemary) extract, Rosmarinus officinalis (rosemary) leaf extract, Thymus serpyllum extract, and Sanguisorba officinalis extract.

3. The adenosine-containing composition according to claim 1, wherein a content by percentage of the component (A) relative to the mass of the adenosine-containing composition is from 5 × 10⁻⁴% by mass to 8% by mass.

4. The adenosine-containing composition according to claim 1, wherein the component (B) is a component which suppresses precipitation of the dissolved component (A).

5. The adenosine-containing composition according to claim 1, wherein the component (B) further contains caffeine.

6. The adenosine-containing composition according to claim 1, wherein a content by percentage of the component (B) relative to the mass of the adenosine-containing composition is 1 × 10⁻⁴% by mass or greater.

7. The adenosine-containing composition according to claim 1, wherein a content by percentage of the component (C) relative to the mass of the adenosine-containing composition is 60% by mass or greater.

8. The adenosine-containing composition according to claim 1, further comprising from 5% by mass to 40% by mass of a polyol relative to the mass of the adenosine-containing composition.

9. The adenosine-containing composition according to claim 1, wherein:
the composition is a single-phase liquid composition; and
in atmospheric pressure at 25° C., the component (A) and the component (B) are dissolved in the adenosine-containing composition.

10. The adenosine-containing composition according to claim 1, comprising substantially no ethanol.

11. The adenosine-containing composition according to claim 1, wherein the composition is applicable in an external-use skin preparation applicable to the skin.

12. The adenosine-containing composition according to claim 1, wherein the composition is an external-use skin preparation for application to the scalp.

13. A method for suppressing precipitation of adenosine, comprising:
dissolving adenosine and a first component to an aqueous solvent containing less than 20% by mass of ethanol, wherein
the first component is at least one selected from the group consisting of salicylic acid and salts thereof, glycyrrhizic acid and salts thereof, and plant extracts.

14. The method according to claim 13, wherein, in the aqueous solvent, a content of water relative to the mass of the aqueous solvent is 60% by mass or greater.

15. The method according to claim 13, wherein the plant extract is at least one selected from the group consisting of Angelica keiskei leaf/stem extract, Prunus armeniaca (apricot) juice, Ginkgo biloba leaf extract, Citrus unshiu peel extract, Scutellaria baicalensis root extract, Panax ginseng root extract, Rosa canina fruit extract, Ononis spinosa extract, Glycyrrhiza glabra (licorice) root extract, Phellodendron amurense bark extract, Sophora angustifolia root extract, Prunus speciosa leaf extract, Hypericum perforatum flower/leaf/stem extract, Crataegus monogyna flower extract, Aesculus hippocastanum (horse chestnut) seed extract, Achillea millefolium extract, Rosa centifolia flower extract, Prunus yedoensis leaf extract, Camellia japonica seed extract, Camellia japonica flower extract, Rubus suavissimus (raspberry) extract, Nelumbo nucifera germ extract, Syzygium jambos leaf extract, Bupleurum falcatum root extract, Melilotus officinalis extract, Centaurea cyanus flower extract, Citrus limon (lemon) extract, Rosmarinus officinalis (rosemary) extract, Rosmarinus officinalis (rosemary) leaf extract, Thymus serpyllum extract, and Sanguisorba officinalis extract.

16. The method according to claim 13, wherein from 5×10⁻⁴% by mass to 8% by mass of adenosine is added to a total mass of composition.

17. The method according to claim 13, wherein the first component is a component which suppresses precipitation of the dissolved adenosine.

18. The method according to claim 13, wherein the first component further contains caffeine.

19. The method according to claim 13, wherein 1 × 10⁻⁴% by mass or greater of the first component is added to the total mass of the composition.

20. The method according to claim 13, further comprising adding, to the aqueous solvent, from 5% by mass to 40% by mass of a polyol relative to the total mass of the composition.

21-22. (canceled)
